(19) United States
(12) Patent Application Publication
(10) Pub. No.: US 2026/0297429 A1
(43) Pub. Date: Oct. 1, 2026

(54) PRODUCTION OF RENEWABLE FUEL FROM NUTRIENT-LADEN WASTE BY A REDUCED-CARBON PROCESS

(71) Applicant: Kore Infrastructure LLC, Bozeman, MT (US)

(72) Inventor: Joseph E. ZUBACK, Camarillo, CA (US)

(21) Appl. No.: 19/475,884

(22) PCT Filed: Apr. 26, 2024
(86) PCT No.: PCT/US24/26474

§ 371 Date: Oct. 16, 2025

Related U.S. Application Data
(60) Provisional application No. 63/462,421, filed on Apr. 27, 2023.

(51) Int. Cl. C10B 53/00; C10B 53/02; C10B 57/10; C10L 3/08; C10L 3/10; C10L 5/42; C10L 5/44; C10L 5/46

(52) U.S. Cl. CPC C10B 53/00; C10B 53/02; C10B 57/10; C10L 3/08; C10L 3/104; C10L 5/42; C10L 5/445; C10L 5/46; C10L 2290/02; C10L 2290/08; C10L 2290/26; C10L 2290/30; C10L 2290/545

(57) ABSTRACT

Described are technologies including processes and system for generation of renewable fuel from nutrient-laden organic waste. The processes described in this specification include processes to convert biomass into a gas using an anoxic gas production process. This output gas can be converted into renewable energy fuels via multiple chemical and/or biological processes.

### BACKGROUND

Modern agriculture produces a large amount of animal wastes, food wastes, and crop residues that have a significant environmental impact. These materials, however, can be used to recover valuable nutrients or energy carriers, e.g., methane using anaerobic digestion technology. Methane is the primary component of natural gas and can be added to or used instead of natural gas in many applications, e.g., as fuel for vehicles, for heating, or a fuel for an engine in an electrical generator to produce electricity. While there are technologies to convert wastes into energy, most are not utilized at the present time due to technical hurdles and unfavorable economics.

### SUMMARY

Described in this specification are technologies including processes and system for generation of renewable fuel from a nutrient-laden organic waste. The processes described in this specification include processes to convert biomass into a gas using an anoxic gas production process. This output gas can be converted into renewable energy fuels via multiple chemical and/or biological processes. The processes can recover nutrients from the biomass that can be used to grown additional biomass. A significant fraction of the atmospheric carbon removed via biomass growth exits as elemental carbon that can be buried or used in applications where the carbon does not return to the atmosphere. This makes the renewable energy fuels produced potentially “carbon negative,” that is, more carbon is sequestered from the atmosphere than released by the process. An example environment where the described technologies can be implemented is a dairy farm, but the technologies can also be applied, e.g., for the use of nutrients to grow “for purpose” crops that are not associated with a dairy operation. These crops include food crops or crops to be used as input for biofuel production. Part of the technologies described in this specification are nutrient recovery processes to remove ammonia from the output gas using an acid scrubber and/or to remove phosphate and potassium from the biochar with an acidification process. These nutrients can be used, e.g., as fertilizer in a biomass growth processes, e.g., to produce input for the gas production process.

In one aspect, this specification describes a method for a renewable fuel production process from a nutrient-laden carbon-based feedstock source by a reduced-carbon process. The method includes receiving a carbon-based feedstock input from a renewable feedstock source, the feedstock source including an agricultural facility or waste treatment facility. The method includes digesting the feedstock input to liberate a first renewable output gas and a digestate. The method includes de-watering the digestate to liberate a first carbonaceous output and a pressate. The method includes extracting a second carbonaceous output from a biomass growth process, the second carbonaceous output including carbon removed from the atmosphere via the biomass growth process, thereby reducing a carbon footprint of the renewable fuel production process. The method includes drying the first carbonaceous output and the second carbonaceous output to generate a dry feed. The method includes processing the dry feed with a gas production process.

The gas production process includes receiving an input fuel stream including a heating gas. The gas production process includes indirectly heating the dry feed with the heating gas in a pyrolyzer via an anaerobic pyrolysis process to produce, from the dry feed, (i) liberated gases including condensable gases and non-condensable gases and (ii) a third carbonaceous output.

The method includes processing the third carbonaceous output with a nutrient recovery process to liberate a first group of nutrients and a pure carbon output. The first group of nutrients includes nutrients from the carbon-based feedstock source. The pure carbon output includes carbon sequestered from the carbon-based feedstock source and the atmosphere via the growth of the biomass, thereby reducing the carbon footprint of the renewable fuel production process. The method includes processing the first renewable output gas with a gas upgrade process to liberate renewable natural gas and processing the liberated gases to liberate an ammonia-free output gas.

### BRIEF DESCRIPTION OF THE DRAWINGS

The novel features of the technologies described in this specification are set forth with particularity in the appended claims. A better understanding of the features and advantages of the present technologies will be obtained by reference to the following detailed description that sets forth illustrative implementations, in which the principles of the technologies are utilized, and the accompanying drawings (also “Figure” and “FIG.” herein), of which:

FIG. 1 is a diagram illustrating a method for producing a renewable gaseous fuel from nutrient-laden waste by a reduced-carbon process, according to an illustrative implementation;

FIG. 2 is a diagram illustrating a method for producing a renewable gaseous fuel from nutrient-laden waste from the reduced-carbon process, including methanation of liberated gases, according to an illustrative implementation; and

FIG. 3 and FIG. 4 are diagrams illustrating methods for producing a renewable gaseous fuel from nutrient-laden waste by a reduced-carbon process, including extracting liberated carbon dioxide gas from the reduced-carbon process to fuel a biomass growth process, according to an illustrative implementation.

### DETAILED DESCRIPTION

The following detailed description represents example modes for carrying out the technologies envisaged. The description is not to be taken in a limiting sense, but is made merely for the purpose of illustrating the general principles.

Described in this specification are technologies for the production of renewable fuel from nutrient-laden (solid) waste by a reduced-carbon process. Renewable fuels have had periods of popularity and periods of disfavor, with their relevance often being tied to the global fossil fuel market. Renewables have generally been considered to have drawbacks including costs of production and overall heating capabilities that are typically lower than traditional hydrocarbons, such as natural gas, octane and other hydrocarbons. The technologies described in this specification can improve efficiencies and reduce costs of renewable fuel production systems.

An example of nutrient-laden waste that can be used as a source (feedstock) for the technologies described in this specification is manure produced by farm animals, e.g., on a dairy farm. Manure is commonly treated in an anaerobic digester to reduce the waste volume and produce biomethane that can be converted into renewable fuel (renewable natural gas). The digestate (digested leftovers) from the anaerobic digester typically possess a high concentration of nutrients and can be used as fertilizer for the growth of food crops. With the increasing size of dairies, there are limited options for nutrient removal and disposal. One method for nutrient removal and disposal is to pyrolyze the de-watered digestate produced by the anaerobic digester under lower temperature conditions to convert the volatiles in the de-watered digestate to produce condensable gases, which are biodegradable. These condensed gases can be sent back to the anaerobic digester to be converted to more biomethane for conversion into renewable natural gas. One drawback of this method is that significant amount of nutrients remains behind in the pressate liquid that is left behind when the digestate is dewatered, and the nutrients are not utilized for the production of renewable fuel.

The technologies described in this specification improve the process for the production of renewable fuel from the nutrient-laden waste by addressing the above-mentioned and other current drawbacks, providing a process that recovers nutrients from both the digestate and pressate and that generates more renewable fuel through a further reduced or even negative carbon footprint. Moreover, the technologies improve the efficiency and reliability of the fuel production processes by reducing the outlay of required capital equipment while at the same time reducing or eliminating the potential impact of, e.g., crop failure or unpredictability of weather.

Described in this specification are technologies for producing a renewable gaseous fuel from nutrient-laden (solid, liquid, or mixed) waste through a reduced carbon footprint process. More particularly, the processes described produce renewable gaseous fuel from an organic feedstock. The renewable fuel is produced with a process implemented to minimize the carbon footprint of the overall process and can be recycled to power the gas production process itself. Byproducts of the renewable fuel can be further harnessed to achieving further efficiencies and reduction in the carbon footprint of the overall process.

The technologies described in this specification integrate production of a renewable gaseous fuel with production of a solid residual containing elemental carbon (e.g., charcoal, char, or biochar) that can be sequestered to prevent return to the atmosphere as carbon dioxide (CO₂). The solid residuals can also be sold commercially, or used as concrete additives, soil amendments, or solid fuel. The input feedstock used with the technologies described in this specification can be solid, liquid, or combinations thereof, e.g., in form of a slurry. The technologies can utilize a wide variety of biogenic carbonaceous organic feedstocks generally considered wastes, such as agricultural wastes, animal manure, high hazard forestry waste, municipal wastewater treatment, plant biosolids, food wastes, demolition wood, and/or non-biogenic carbonaceous feedstocks, such as waste plastics and tires, that contain biogenic components.

The efficiency of the renewable gas production carried out according to the methods described in this specification can be significantly higher than production involving the use of either natural gas or solar thermal energy for nutrient removal and disposal. This efficiency can be achieved because of the availability of abundant waste materials that are suitable feed sources for production of renewable gas, the multi-functional use of the carbonaceous solid byproduct as a fuel, and the overall beneficial environmental impact of using a renewable fuel to replace a fossil fuel (particularly by reducing the carbon intensity of transportation fuels).

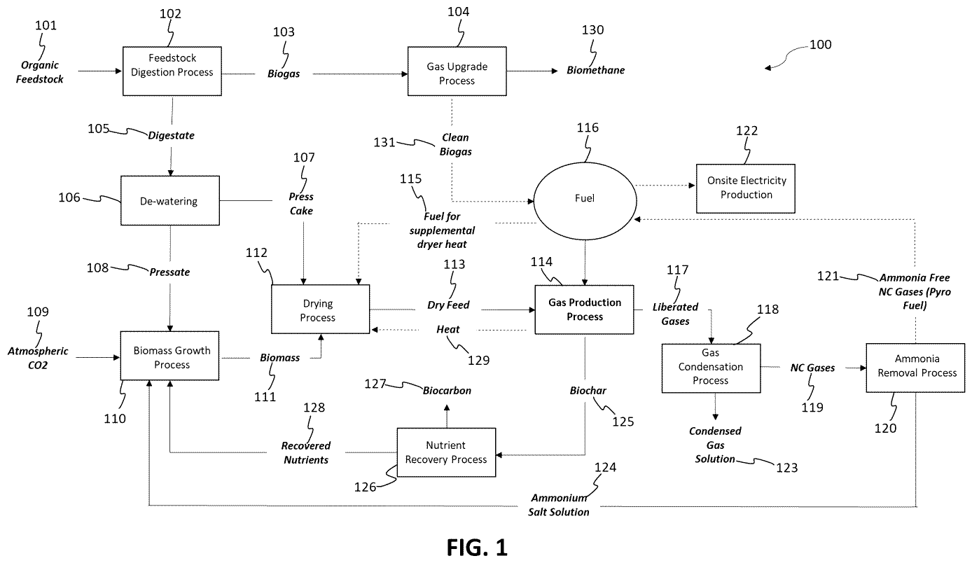

FIG. 1 illustrates a process 100 of producing a renewable gaseous fuel 130 from nutrient-laden waste. Process 100 includes a gas production process 114 as described below in this specification. The gas production process 114 (e.g., using a pyrolyzer) receives as input a dry feed 113 derived at least in part from a digestate 105 produced from digesting organic feedstock (e.g., dairy manure) 101 via a feedstock digestion process 102. Gas production process 114 receives a fuel stream 116 to heat the dry feed 113 to produce liberated gases 117 and residual carbonaceous solid output 125 (e.g., biochar). The process 100 also includes a feedstock digestion process 102, which liberates a renewable gas (e.g., biogas) 103, which in turn is processed via a gas upgrade process 104 to liberate a renewable fuel output (e.g., biomethane) 130 and a second renewable gas (e.g., clean biogas 131). Clean biogas 131 can be used as part of the fuel stream 116 for the gas production process 114. A portion of fuel stream 116 can also be used as fuel for an onsite electricity production process 122. The process 100 also includes a gas condensation process 118 to produce a condensed gas solution 123 and an ammonia removal process 120 to further liberate ammonia free non-condensable (NC) gases 121 and a nutrient rich solution (e.g., ammonium salt solution) 124. Non-condensable (NC) gases 121 can be used as part of the fuel stream 116 for gas production process 114. The nutrient rich solution (e.g., ammonium salt solution) 124 can be used as fertilizer for a biomass growth process 110, as described further below. In some implementations, the condensed gas solution 123 is processed on site, e.g., at the same facility as the gas production process 114. In some implementations, the condensed gas solution 123 is processed in a centralized location or facility (not shown in figure) to liberate the renewable fuel output (e.g., biomethane) 130 and/or other renewable fuel outputs (e.g., hydrogen gas and hydrocarbons). The centralized location or facility can process the condensed gas solution from multiple gas production processes 114, e.g., multiple gas production processes 114 at separate locations.

The organic feedstock 101 provides input for a feedstock digestion process 102 to produce the digestate 105 that is processed and converted into a dry feed 113 for the gas production process 114. The organic feedstock 101 includes carbon-based material and can be selected from a variety of biogenic carbonaceous feedstocks generally considered wastes, such as agricultural wastes, animal manure, high hazard forestry waste, municipal wastewater treatment plant biosolids, food wastes, demolition wood, and/or non-biogenic carbonaceous feedstocks, such as waste plastics and tires that contain biogenic components. In some implementations, the feedstock digestion process 102 includes a bacterial digestion process. The digestate 105 produced by the feedstock digestion process 102 is processed via a de-watering process 106 to produce a (solid) press cake 107 and a liquid pressate 108. In some implementations, the de-watering process 106 includes de-watering the digestate 105 with a screw press. In some implementations, the de-watering process 106 includes de-watering the digestate 105 with a mechanical press. The liquid pressate 108 is rich in nutrients and can also be used for a biomass growth process 110 to generate biomass 111. The biomass growth process 110 utilizes atmospheric CO₂ 109, thereby reducing the overall carbon footprint of the process 100. The press cake 107 and biomass 111, each alone or in combination, provide input for a drying process 112 to generate at least a portion of dry feed 113 for the gas production process 114. In some implementations, the process 100 further includes utilizing a portion of the fuel stream 116 as supplemental fuel 115 for the drying process 112. In some implementations, heat 129 generated from the gas production process 114 is utilized in the drying process 112. In some implementations, solar energy or other sources of energy can be utilized in the drying process 112.

The dry feed 113 is used in a gas production process 114 that is generally anoxic, i.e., typically involving an anoxic heating process. In general, gas production process 114 is executed at a temperature that generates the liberated gases 117 and the residual carbonaceous solid (e.g., biochar) 125. The liberated gases 117 have sufficient calorific value that can be harvested and used as part of the fuel stream 116 for the gas production process 114 (or at least a portion thereof). As illustrated in the figures, harvesting and using the liberated gases 117 and extracting the residual carbonaceous solid 125 serves to reduce the carbon footprint of the overall process. That reduction can be further enhanced by recycling the liberated gases 117 into the gas production process 114.

Gas production process 114 includes a pyrolysis process, e.g., using a pyrolizer. In a pyrolysis process used in the technologies described in this specification, the feedstock is not in direct contact with a flame. Heating of the input or feed material is accomplished by applying an external heat source without oxygen under anaerobic conditions (anoxic) to prevent combustion of the (solid) input material. In some implementations, hot gas can be produced in an external burner to transfer heat to the feedstock through the walls of pipes (retorts) through which the feedstock continuously travels, liberating gasses when the feedstock reaches pyrolysis temperatures. In some implementations, an example burner can use natural gas as a fuel, e.g., from fuel stream 116. In some implementations, an example burner can use biogas, e.g., biogas 131, as a fuel. In some implementations, an example burner can use ammonia free NC gases 121 as a fuel, which are a product of the gas production process 114, as described below. In some implementations, an example burner can use a combination of natural gas and biogas, or a combination of natural gas and NC gas, or a combination of biogas and NC gas, or a combination of natural gas, biogas, and NC gas as fuel.

The pyrolysis may occur over a range of temperatures, the optimal temperature being selected as needed to liberate sufficient combustible gas from the dry feed 113. The temperature may be up to about 800° C. The temperature may be between about 400° C. and about 800° C., or between about 450° C. and about 750° C. The temperature may be between about 500° C. and about 700° C. The temperature may be about 600° C.

The pyrolysis process can also occur over a range of heating rates, the optimal rate being selected in conjunction with the desired temperature based on the selected input (e.g., dry feed 113 derived from organic feedstock 113). In some implementations, the heating rate is between about 4° C./min and about 12° C./min. In some implementations, the heating rate is between about 7° C./min and about 9° C./min. In some implementations, the heating rate of the pyrolysis is about 8° C./min. Other methods of gas production can be used (e.g., combustion, carbonization, charring, devolatilization) with similar or identical temperatures and heating rates to the pyrolysis conditions discussed above.

As discussed above, the gas production process 114 receives as an input the fuel stream 116, which can include natural gas. The fuel stream 116 can combine various recycle streams or other inputs as the final heating gas input to the gas production process 114. By utilizing recycle streams (e.g., the clean biogas 131 liberated during the gas upgrade process 104) as a component of the fuel stream 116 for the gas production process 114, the efficiency of the process 100 is further increased, and the carbon footprint of the method 100 is further reduced. In some implementations, the gas upgrade process 104 can be implemented to remove soot particles and non-desirable gases, such as acidic gases like hydrogen sulfide, hydrogen chloride, hydrogen fluoride, ammonia, volatilized metals, carbon dioxide, or other undesirable gases that condense into liquids or reduce the heat value of the gas to produce the clean gas biogas 131 to be used part of the fuel stream 116 for the gas production process 114.

As discussed above, the gas production process 114 generates the liberated gases 117 and the residual carbonaceous solid (e.g., biochar) 125. The residual carbonaceous solid 125 can be further refined in a nutrient recovery process 126 to yield a second residual carbonaceous solid 127 (e.g., biocarbon) and to recover nutrients 128. In some implementations, the nutrient recovery process 126 can include, but is not limited to, an acidification process, an ash removal process, or an ammonia removal process. In some implementations, the acidification process can further include scrubbing with at least one of hydrochloric acid, sulfuric acid, or nitric acid. The recovered nutrients 128 can include, but are not limited to, potassium and phosphate. The recovered nutrients 128 can be used, e.g., as fertilizer for the biomass growth process 110.

As discussed above, the liberated gases 117 are the volatile gases liberated by the gas production process 114 containing a mixture of condensable and non-condensable gases. The liberated gases 117 are subsequently processed with a gas condensation process 118 to separate the NC gases 119 from a condensed gas solution 123 containing the condensable gases. The NC gases 119 are then processed via the ammonia removal process 120 to liberate ammonia-free NC gases 121 (e.g., pyro-fuel) and the nutrient rich solution 124 (e.g., ammonium salt solution). The liberated ammonia-free NC gases 121 can also be used as part of the fuel stream 116 for the gas production process 114. The liberated nutrient rich solution 124 can also be used (as fertilizer) for the biomass growth process 110. In some implementations, the nutrient rich solution 124 includes at least one of ammonium sulfate, ammonium chloride, or ammonium nitrate.

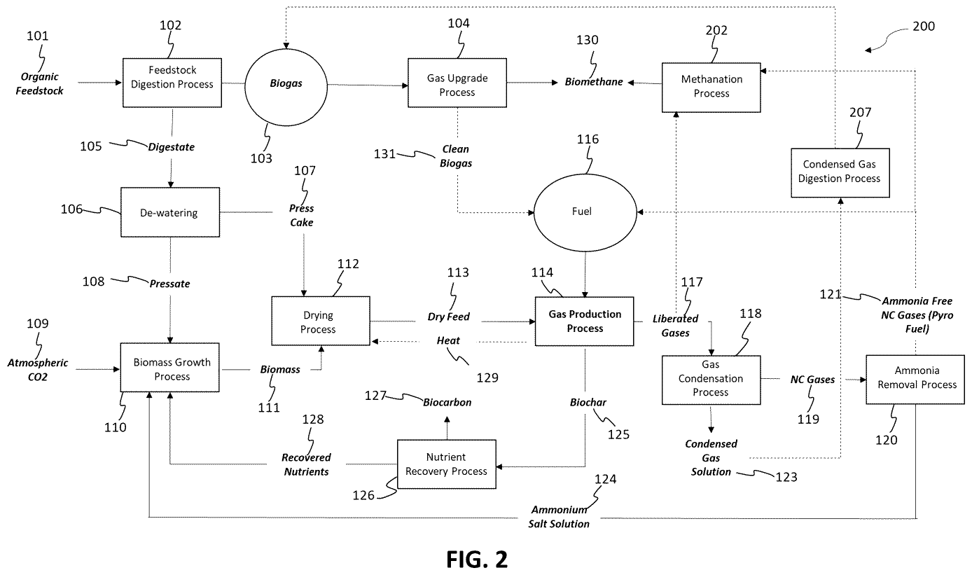

FIG. 2 illustrates a process 200 for producing a renewable gaseous fuel from the nutrient-laden waste including a methanation process 202 for converting carbon monoxide and carbon dioxide gases into biomethane through a hydrogenation process. Process 200 can include any and all elements of process 100 (e.g., supplemental fuel 115 and/or onsite electricity production process 122, not shown). As described above, processing the liberated gases 117 generated by gas production process 114 with the gas condensation process 118, and the ammonia removal process 120 generates the condensed gas solution (containing condensed gases) 123 and the ammonia-free NC gases 121. In an implementation, the condensed gas solution 123 can be processed by the feedstock digestion process 102 to liberate additional renewable gas (e.g., biogas) 103. In an implementation of the process 200, the condensed gas solution 123 is further processed via a condensed gas digestion process 207 to liberate additional renewable gas (e.g., biogas) 103, which is further processed via the gas upgrade process 104 to liberate the renewable fuel output (e.g., biomethane) 130, thereby increasing the efficiency of the process 200. In this implementation, the condensed gas digestion process 207 is separate from the feedstock digestion process 102 because the condensed gases in the condensed gas solution 123 may negatively impact the feedstock digestion process 102 by adversely impacting microbial conditions of the feedstock digestion process 102. In an implementation of the process 200, a portion of the liberated gases 117, or a portion of the ammonia-free NC gases 121, or a combination thereof, are processed with the methanation process 202. In the methanation process 202, an amount of carbon monoxide and carbon dioxide gases from a portion of the liberated gases 117, a portion of the ammonia-free NC gases 121, or a combination thereof, are hydrogenated to produce additional renewable fuel output (e.g., biomethane) 130, thereby also increasing the efficiency of the process 200 and further reducing the fuel cost and the carbon footprint of process 200.

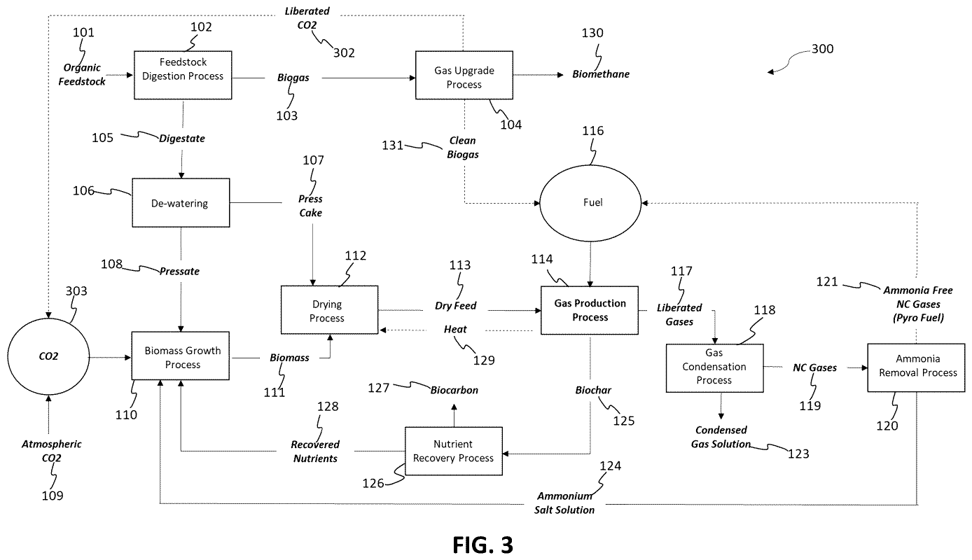

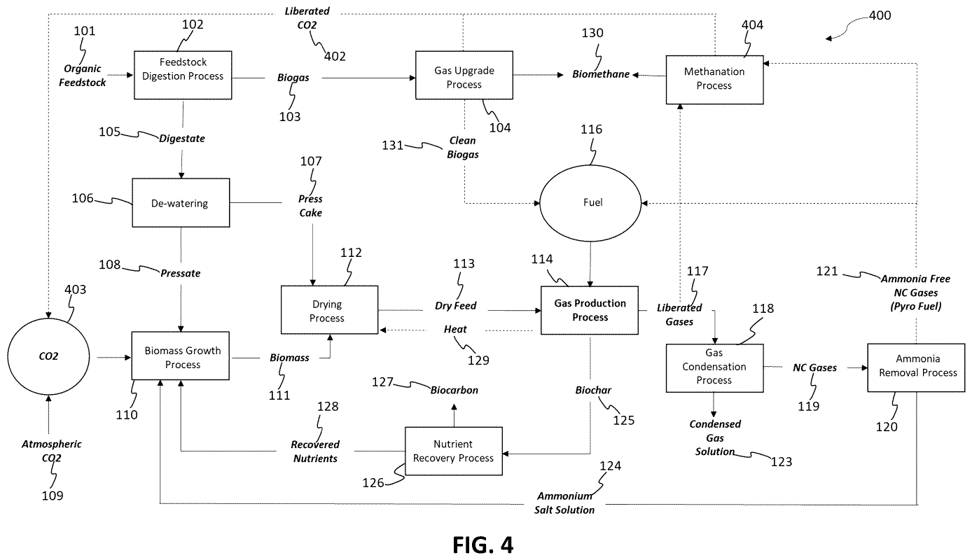

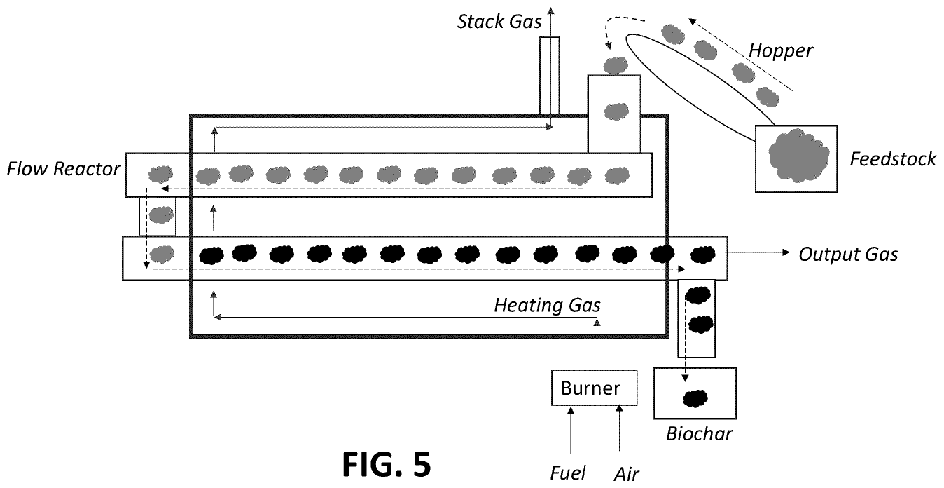

FIG. 3 and FIG. 4 illustrate processes 300 and 400 for producing a renewable gaseous fuel from a nutrient-laden waste by a reduced-carbon process as described above, enhanced with extracting liberated carbon dioxide gas 302/402, e.g., to fuel the biomass growth process. Processes 300 and 400 can include any and all elements of processes 100 and 200 (e.g., supplemental fuel 115 and/or onsite electricity production process 122, not shown). As referenced above in FIGS. 1 and 2, the feedstock digestion process 102 digests the organic feedstock 101 to liberate the renewable gas (e.g., biogas) 103, which is processed via the gas upgrade process 104 to liberate the renewable fuel output (e.g., biomethane) 130. In process 300 illustrated in FIG. 3, the gas upgrade process 104 is modified to further process the renewable gas 103 to generate liberated CO₂ 302. The liberated CO₂ 302 can be combined with the atmospheric CO₂ 109 in a CO₂ source 303 to be used for the biomass growth process 110. This process further reduces the carbon footprint of the process 300, generating more biomass 111 to increase the renewable fuel source 130 output of the method 300, thereby increasing the overall efficiency of the process.

As discussed above and illustrated in FIG. 2, a portion of the liberated gases 117, and/or a portion of the ammonia-free NC gases 121 are processed with the methanation 202 process to yield additional renewable fuel output (e.g., biomethane) 130, increasing efficiency of the process 200 and further reducing the fuel cost and the carbon footprint of the process 200. As further discussed in FIG. 2, the condensed gas solution 123 is further processed via condensed gas digestion process 207 to liberate additional renewable gas (e.g., biogas) 103, which is further processed via the gas upgrade process 104 to liberate the renewable fuel output (e.g., biomethane) 130, thereby increasing the efficiency of the process 200. The methanation process 404 of FIG. 4 is modified to further process the portion of the liberated gases 117, and/or a portion of the ammonia-free NC gases 121 to liberate an amount of CO₂ 402. A portion of CO₂ from liberated gases 117, and/or a portion of ammonia-free NC gases 121 are used for hydrogenation to produce additional renewable fuel output (e.g., biomethane). FIG. 4 also includes the modified gas upgrade process 104, e.g., as illustrated in FIG. 3, for further processing the renewable gas 103 to generate liberated CO₂ 402. The liberated CO₂ 402 can be combined with the atmospheric CO₂ 109 in a CO₂ source 403 to be used for the biomass growth process 110. This process further reduces the carbon footprint of the process 400, generating more biomass 111 to increase the renewable fuel source 130 output of the process 400, further increasing the overall efficiency of the process.

The foregoing is merely illustrative of the principles of the disclosure, and the apparatuses can be practiced by other than the described implementations, which are presented for purposes of illustration and not of limitation. It is to be understood that the technologies disclosed herein, while shown for use, e.g., in dairy production, may be applied to systems to be used in other systems. Variations and modifications will occur to those of skill in the art after reviewing this disclosure. The disclosed features may be implemented, in any combination and sub-combination (including multiple dependent combinations and sub-combinations), with one or more other features described herein. The various features described or illustrated above, including any components thereof, may be combined or integrated in other systems. Moreover, certain features may be omitted or not implemented. Examples of changes, substitutions, and alterations are ascertainable by one skilled in the art and could be made without departing from the scope of the information disclosed herein. All references cited herein are incorporated by reference in their entirety and made part of this application.

### Example Implementations

Item 1. A method for a renewable fuel production process from a nutrient-laden carbon-based feedstock source by a reduced-carbon process, the method comprising: receiving a carbon-based feedstock input from a renewable feedstock source, the feedstock source comprising an agricultural facility or a waste treatment facility; digesting the feedstock input to liberate first renewable output gas and a digestate; de-watering the digestate to liberate a first carbonaceous output and a pressate; extracting a second carbonaceous output from a biomass growth process, the second carbonaceous output comprising carbon removed from the atmosphere via the biomass growth process, thereby reducing a carbon footprint of the renewable fuel production process; drying the first carbonaceous output and the second carbonaceous output to generate a dry feed; processing the dry feed with a gas production process, the gas production process comprising: receiving an input fuel stream comprising a heating gas; indirectly heating the dry feed with the heating gas in a pyrolyzer via an anaerobic pyrolysis process to produce, from the dry feed, (i) liberated gases comprising condensable gases and non-condensable gases and (ii) a third carbonaceous output; processing the third carbonaceous output with a nutrient recovery process to liberate a first group of nutrients and a pure carbon output, where: the first group of nutrients comprises nutrients from the carbon-based feedstock source; and the pure carbon output comprises carbon sequestered from the carbon-based feedstock source and the atmosphere via the growth of the biomass, thereby reducing the carbon footprint of the renewable fuel production process; processing the first renewable output gas with a gas upgrade process to liberate renewable natural gas; and processing the liberated gases to liberate an ammonia-free output gas.

Item 2. The method of item 1, wherein processing the liberated gases comprises: separating the condensable gases and non-condensable gases; and processing the non-condensable gases with an ammonia removal process to liberate the ammonia-free output gas and a second group of nutrients, the second group of nutrients comprising an ammonium salt solution.

Item 3. The method of item 2, comprising: processing the condensable gases with a gas condensation process to liberate a condensed gas solution.

Item 4. The method of item 3, comprising: processing the condensed gas solution with a condensed gas digestion process to liberate a first renewable output gas.

Item 5. The method as in any one of items 2-4, comprising: processing the ammonia-free output gas or the liberated gases, or both, with a methanation process to produce biomethane.

Item 6. The method as in any one of items 2-5, comprising using one or both of (i) the first group of nutrients or (ii) the second group of nutrients as input for the biomass growth process.

Item 7. The method as in any one of items 1-6, wherein the nutrient recovery process comprises at least one of: a) an acidification step; b) an ash removal step; or c) an ammonia removal step.

Item 8. The method of item 7, wherein the acidification step comprises scrubbing the non-condensable gases with at least one of hydrochloric acid, sulfuric acid, or nitric acid.

Item 9. The method as in any one of items 1-8, wherein: the first group of nutrients comprises potassium and phosphate; and the ammonium salt solution comprises at least one of ammonium sulfate, ammonium chloride, or ammonium nitrate.

Item 10. The method as in any one of items 1-9, wherein the digesting the feedstock input comprises a bacterial digestion process.

Item 11. The method as in any one of items 1-10, wherein the gas upgrade process comprises a carbon dioxide (CO₂) removal process.

Item 12. The method as in any one of items 1-11, wherein the gas upgrade process further comprises liberating a clean biogas comprising hydrogen gas.

Item 13. The method of item 12, wherein the input fuel stream of the gas production process comprises the clean biogas.

Item 14. The method as in any one of items 1-13, wherein the input fuel stream of the gas production process comprises a portion of the ammonia-free output gas.

Item 15. The method as in any one of items 1-14, wherein the input fuel stream of the gas production process comprises natural gas.

Item 16. The method of item 12, wherein the input fuel stream of the gas production process comprises at least two of the clean biogas, a portion of the ammonia-free output gas, and natural gas.

Item 17. The method as in any one of items 1-16, wherein the digestate is processed with a screw press for de-watering the digestate.

Item 18. The method as in any one of items 1-16, wherein the digestate is processed with a mechanical press for de-watering the digestate.

Item 19. The method as in any one of items 1-18, wherein the drying the first carbonaceous output and the second carbonaceous output comprises drying with solar energy.

Item 20. The method as in any one of items 1-19, wherein the drying the first carbonaceous output and the second carbonaceous output comprises drying with heat generated from the gas production process.

Item 21. The method as in any one of items 1-20, wherein the carbon-based feedstock input is a carbon-based feedstock input.

Item 22. The method as in any one of items 1-20, wherein the carbon-based feedstock input comprises manure and/or other biodegradable organic waste.

Item 23. The method as in any one of items 1-22, further comprising using the pressate in the biomass growth process.

Item 24. The method as in any one of items 1-23, further comprising liberating carbon dioxide (CO₂) from the first renewable output gas with the gas upgrade process.

Item 25. The method as in any one of items 5-24, further comprising liberating CO₂ from the ammonia-free output gas with the methanation process.

Item 26. The method as in any one of items 24-25, comprising removing the liberated CO₂ via the biomass growth process, thereby further reducing the carbon footprint of the renewable fuel production process.

1-26. (canceled)

27. A method for a renewable fuel production process from a nutrient-laden carbon-based feedstock source by a reduced-carbon process, the method comprising:
receiving a feedstock input from a carbon-based feedstock source, the feedstock source comprising an agricultural facility or a waste treatment facility;
digesting the feedstock input to liberate a first renewable output gas and a digestate;
de-watering the digestate to liberate a first carbonaceous output and a pressate;
extracting a second carbonaceous output from a biomass growth process, the second carbonaceous output comprising a carbon removed from an atmosphere via the biomass growth process, thereby reducing a carbon footprint of the renewable fuel production process;
drying the first carbonaceous output and the second carbonaceous output to generate a dry feed;
processing the dry feed with a gas production process, the gas production process comprising:
receiving an input fuel stream comprising a heating gas;
indirectly heating the dry feed with the heating gas in a pyrolyzer via an anaerobic pyrolysis process to produce, from the dry feed, (i) liberated gases comprising condensable gases and non-condensable gases and (ii) a third solid carbonaceous output;
processing the third carbonaceous output with a nutrient recovery process to liberate a first group of nutrients and a pure carbon output, wherein:
the first group of nutrients comprises one or more nutrients from the carbon-based feedstock source; and
the pure carbon output comprises carbon sequestered from the carbon-based feedstock source and the atmosphere via the growth of the biomass, thereby reducing a carbon footprint of the renewable fuel production process;
processing the first renewable output gas with a gas upgrade process to liberate renewable natural gas; and
processing the liberated gases to liberate an ammonia-free output gas.

28. The method of claim 27, wherein processing the liberated gases comprises:
separating the condensable gases and non-condensable gases; and
processing the non-condensable gases with an ammonia removal process to liberate the ammonia-free output gas and a second group of nutrients, the second group of nutrients comprising an ammonium salt solution.

29. The method of claim 28, comprising:
processing the condensable gases with a gas condensation process to liberate a condensed gas solution.

30. The method of claim 29, comprising:
processing the condensed gas solution with a condensed gas digestion process to liberate a first renewable output gas.

31. The method of claim 30, comprising:
processing the ammonia-free output gas or the liberated gases, or both, with a methanation process to produce biomethane.

32. The method of claim 31, comprising using one or both of (i) the first group of nutrients or (ii) the second group of nutrients as input for the biomass growth process.

33. The method of claim 32, wherein the nutrient recovery process comprises at least one of:
an acidification step;
an ash removal step; or
an ammonia removal step.

34. The method of claim 33, wherein the acidification step comprises scrubbing the non-condensable gases with at least one of hydrochloric acid, sulfuric acid, or nitric acid.

35. The method of claim 34, wherein:
the first group of nutrients comprises potassium and phosphate; and
the ammonium salt solution comprises at least one of ammonium sulfate, ammonium chloride, or ammonium nitrate.

36. The method of claim 35, wherein the digesting the feedstock input comprises a bacterial digestion process.

37. The method of claim 36, wherein the gas upgrade process comprises a carbon dioxide (CO₂) removal process.

38. The method of claim 37, wherein the gas upgrade process further comprises liberating a clean biogas comprising hydrogen gas.

39. The method of claim 38, wherein the input fuel stream of the gas production process comprises the clean biogas.

40. The method of claim 39, wherein the input fuel stream of the gas production process comprises a portion of the ammonia-free output gas.

41. The method of claim 40, wherein the input fuel stream of the gas production process comprises natural gas.

42. The method of claim 38, wherein the input fuel stream of the gas production process comprises at least two of the clean biogas, a portion of the ammonia-free output gas, and natural gas.

43. The method of claim 42, wherein the digestate is processed with a screw press for de-watering the digestate.

44. The method of claim 42, wherein the digestate is processed with a mechanical press for de-watering the digestate.

45. The method of claim 43, wherein the drying the first carbonaceous output and the second carbonaceous output comprises drying with solar energy.

46. The method of claim 45, wherein the drying the first carbonaceous output and the second carbonaceous output comprises drying with heat generated from the gas production process.

47. The method of claim 46, wherein the carbon-based feedstock input is a carbon-based feedstock input.

48. The method of claim 46, wherein the carbon-based feedstock input comprises manure and/or other biodegradable organic waste.

49. The method of claim 48, further comprising using the pressate in the biomass growth process.

50. The method of claim 49, further comprising liberating carbon dioxide (CO₂) from the first renewable output gas with the gas upgrade process.

51. The method of claim 50, further comprising liberating CO₂ from the ammonia-free output gas with the methanation process.

52. The method of claim 51, comprising removing the liberated CO₂ via the biomass growth process, thereby further reducing the carbon footprint of the renewable fuel production process.

53. A system for a renewable fuel production process from a nutrient-laden carbon-based feedstock source by a reduced-carbon process, the system comprising:
a renewable feedstock source generating a carbon based feedstock, the renewable feedstock source comprising a biomass from a biomass growth process or carbonaceous waste residuals from a waste treatment facility;
an input device configured to receive the biomass from a biomass growth process
a flow reactor device configured as a continuous flow reactor characterized by heating of the input to temperatures necessary for release of gases, the flow reactor device coupled to the input device, the flow reactor device characterized by a pyrolysis process, configured to
thermally decompose the feedstock input to liberate a first renewable output gas and a solid carbon and nutrient containing material comprising a first carbonaceous output, configured to extract a second carbonaceous output from the first renewable output gas such that the second carbonaceous output consisting of gaseous carbon entities comprised of carbon removed from an atmosphere using a biomass growth process to reduce a carbon footprint of a renewable fuel production process;
a thermal source generating a heating gas configured with the continuous flow reactor such that the heating gas transfers a heat through an enclosure in an indirect manner and without physical contact to the carbon based feedstock, the heating gas having a flow direction opposite of a direction of the carbon based feedstock traversing through the continuous flow reactor;
a fuel source coupled to the thermal source, the fuel source comprising a natural gas, a recycled gas, or a combustible fuel; and
an output device coupled to flow reactor device generating a biochar material derived from the first carbonaceous output and the second carbonaceous output and a biogas mixture comprising a carbon dioxide, carbon monoxide, a hydrogen, a water, and one or more trace gases.
